(19) United States
(12) Patent Application Publication
(10) Pub. No.: US 2004/0059264 A1
(43) Pub. Date: Mar. 25, 2004

(54) Footprint analyzer

(76) Inventors: Kunihiko Nishibe, Amagasaki (JP); Masao Otani, Amagasaki (JP); Akiteru Takagi, Niigata (JP)

Correspondence Address: WENDEROTH, LIND & PONACK, L.L.P., 2033 K STREET N. W., SUITE 800, WASHINGTON, DC, 20006-1021, US

(21) Appl. No.: 10/662,304

(22) Filed: Sep. 16, 2003

(30) Foreign Application Priority Data
Feb. 19, 2002 (JP) 2003-40940

(51) Int. Cl. A61B 5/103; A61B 5/117

(52) U.S. Cl. 600/587; 600/592

(57) ABSTRACT

The present invention provides a footprint analyzer capable of recording walking exercise data, quantitatively grasp the situation of exercise, and applying the exercise data to diagnosing system by comparing them with past data.

For that purpose, this footprint analyzer comprises an image fetching unit for fetching walking images, an image processing unit for treating the images from this image fetching unit, a display unit for displaying the results of operation of this image processing unit, and a storage unit for saving the results of operation, the image processing unit discriminating the left and right legs from the fetched images and calculating the floor face contact position of the respective legs and chronological changes of each leg at the floor face contact position, and the display unit displaying the floor face contact position of the respective legs and chronological changes of each leg.

### BACKGROUND OF THE INVENTION

The present invention concerns a footprint analyzer fit for use by aged persons, persons disabled in legs and loins, patients in rehabilitation after medical operation of legs and loins, etc., more specifically a footprint analyzer capable of recording exercise data.

Walking exercise is often adopted for the rehabilitation of aged persons, persons disabled in legs and loins or patients after medical operation, etc., and especially a parallel-bar type walking device is frequently used as instrument for supporting this exercise. This parallel-bar type walking device consists of a pair of parallel bars disposed about horizontally in the shape of a handrail on stays, and is designed for the user to train his/her legs and loins, by walking while gripping those parallel bars.

On the other hand, footprint analyzer is an instrument used on the occasion of such walking exercise, and a conventional footprint analyzer was intended to analyze, after walking, the footprint position from the position of the powder sticking to the feet or shoes, analyze, after walking, the footprint position by using walking position detecting sensors disposed on the entire floor surface, analyze walking behavior of a walker with infrared camera, by attaching a marker which reflects infrared rays to the feet, or analyze the footprint position by means of a floor reaction meter detecting foot force provided on the floor.

However, said conventional footprint analyzer has problems such as troublesomeness of applying powder to the sole of feet or attaching a marker to the feet, etc., high price of walking position detecting sensors disposed on the entire floor surface, expensive behavior analysis of markers by infrared-ray camera due to simultaneous use with floor reaction meter, etc. and, for those reasons, the diffusion rate of this analyzer is substantially rather low under the current situation.

Therefore, even if walking exercise is implemented, it was rather difficult to grasp any quantitative exercise data regarding the walking behavior, thus presenting a problem of difficulty of making any quantitative determination, at the level of doctors and/or physical therapists.

### SUMMARY OF THE INVENTION

The objective of the present invention, realized in view of said problems of conventional footprint analyzers, is to provide a footprint analyzer capable of recording walking exercise data, quantitatively grasping the exercise situation, and applying the exercise data to a diagnostic system through comparison of past data.

To achieve said objective, the footprint analyzer according to the present invention is comprised of an image fetching unit for retrieving walking images, an image processing unit for treating the images from the image fetching unit, a display unit for displaying the results of the operation of the image processing unit, and a storage unit for saving the operation results, and is characterized in that said image processing unit discriminates the left and right legs from said fetched images and calculates the floor face contact position of the respective legs and the chronological changes of each leg at the floor face contact position, while said display unit displays the floor face contact position of the respective legs and chronological changes of each leg.

This footprint analyzer, in which said image processing unit discriminates the left and right legs from said fetched images and calculates the floor face contact position of the respective legs and the chronological changes of each leg at the floor face contact position, while said display unit displays the floor face contact position of each the left and right legs and chronological changes of each leg, makes it possible for the person doing a walking exercise to recognize the behavior of both his/her left and right legs, yet walk while visually checking with his/her own eyes his/her own walking posture, walking foot position, etc.

In such a case, said image processing unit performs operation or statistical processing of the walker's walking speed, the distance between the left and right legs, the distance between the front and rear legs, the distance between the front and rear positions of each leg, any staggering to the left or right, the support rate of both legs and/or the support rate of a single leg, and said display unit displays part or all of the operation results.

This makes it possible for the person doing walking exercises to walk while visually checking with his/her own eyes his/her own walking posture, namely inclination of the legs and knees, inclination to the left or right or in some cases, waist balance, and the smoothness of walking, etc.

Moreover, said storage unit memorizes the walker's symbol, the operation results or the results of statistically processing the walking speed, the distance between the left and right legs, the distance between the front and rear legs, the distance between the front and rear positions of each leg, any staggering to the left or right, the support rate of both legs and/or the support rate of a single leg of the walker at each time of measurement, and said image processing unit can perform a comparative operation of data for each measurement.

As a result, it becomes possible to quantitatively grasp the progress of the exercise and the degree of recovery, etc., through comparison with past data, etc., and this enables doctors and physical therapists to effectively promote walking exercise for patients by effectively utilizing the results together with said data, to realize a short-period walking exercise.

Furthermore, said display unit may be provided in front direction of the walker.

This makes it possible for the walker to perform walking exercises while looking straight ahead, without looking downward.

Furthermore, the image processing unit accumulates the right step on one of the coordinate axes of two-dimensional coordinates, accumulates the left step on the other axis of coordinate, determines the coordinates of the respective steps, and indicates the coordinates on a coordinate graph.

This makes it possible for the person performing walking exercises, etc. to know which of his/her legs is unusual and what the degree of abnormality is at a glance by looking at the coordinate graph, because, in a case where there is something wrong with one of the legs, the step of the leg on the opposite side generally becomes short and, as the cumulative value of this step gradually becomes shorter, the coordinates for each step will be indicated through the standard coordinates of sound people, in correspondence to the amount of difference between the left and right steps.

Also, the image processing unit can display images by turning them at required angle, so that the standard coordinates of a sound person may be given on the axis of ordinate.

This makes it easier to recognize unusual leg sides and the degree of abnormality.

Plus, the image processing unit can display the center locus for the respective position of each leg.

This makes it easy to intuitively grasp the state of unevenness left or right of the center position between the left and right sides of the walker. Namely, one can understand, at a glance, if the walker is walking straight or on a curved line.

### BRIEF DESCRIPTION OF THE DRAWINGS

FIG. 1 is a perspective view showing an embodiment of the footstep analyzer according to the present invention.

FIG. 2 is a drawing showing an example of image fetched through the image fetching unit on the footstep analyzer.

FIG. 3 is a drawing continuously indicating the landing position of each leg after a walk.

FIG. 4 (a) is a graph showing the situation of walking of a sound person, while (b) is a graph showing the situation of walking of a handicapped person.

FIG. 5 indicates the contents of FIG. 4 by showing the axis of ordinate upside down, (a) being a graph showing the situation of walking of a sound person, and (b) a graph showing the situation of walking of a handicapped person.

FIG. 6 indicates an example of showing the coordinates of the respective steps, by accumulating the left and right steps on coordinate axes, (a) being a graph showing the situation of walking of a sound person, (b) of a person paralyzed on the left half, and (c) of a person paralyzed on the right half.

FIG. 7 shows the contents of FIG. 6 by turning the graphs counterclockwise by 45.

FIG. 8 is an explanatory drawing of a case where the contents of the coordinate graph in FIG. 7 are converted into actual physical quantities.

FIG. 9 is a drawing showing an example of display on the display unit.

FIG. 10 is a perspective view showing an example in which the image fetching unit is disposed on the back face side of the person taking walking exercise.

FIG. 11 is a drawing showing an example of display on the display unit of the footstep analyzer indicated in FIG. 10.

FIG. 12 is a chart showing the data processing flow.

### DETAILED DESCRIPTION OF THE INVENTIONS

An embodiment of the footprint analyzer according to the present invention will be explained hereafter with reference to drawings.

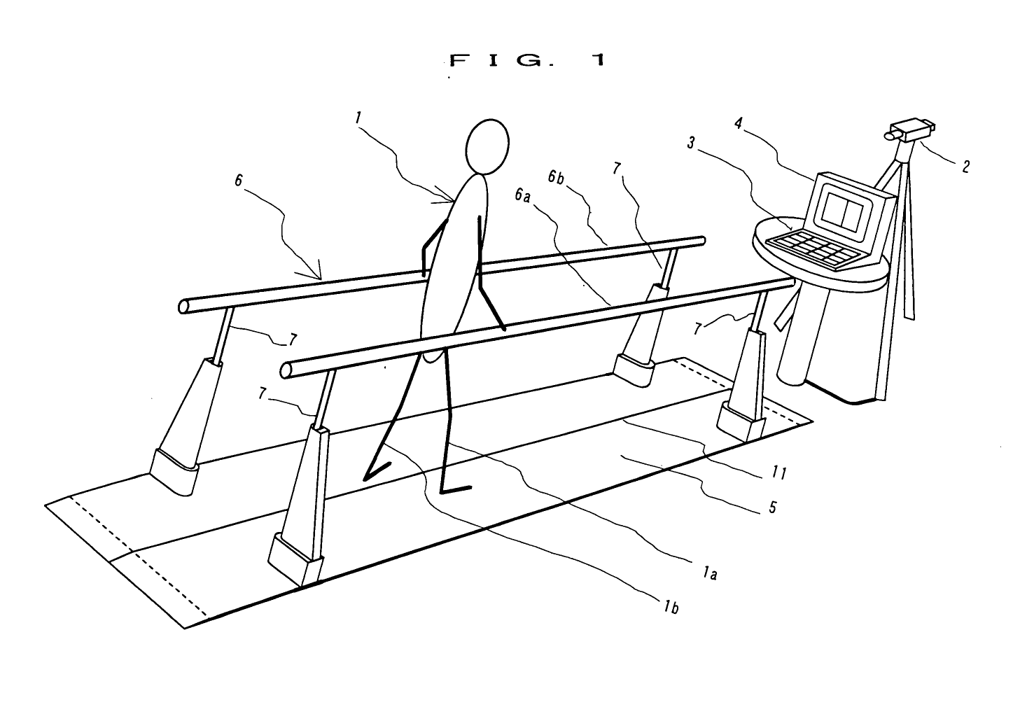

FIG. 1 is a perspective view showing an embodiment of the present invention, 1 indicating a walker, 1a the right leg, 1b the left leg, and 2 an image fetching unit consisting of a video camera, etc.

This footprint analyzer fetches the walking behavior of the walker 1 from about the front direction by means of an image fetching unit 2, electronically transmits the fetched image to an image processing system 3 such as personal computer, etc., analyzes the walking footprints by means of an image processing unit incorporated in the image processing system 3, and indicates the results of operation on a display unit 4.

The results of operation of the image processing unit are memorized by a storage unit incorporated in the image processing system.

In the drawing, 5 indicates the walking floor face, and 6 represents parallel bars, i.e. a pair of parallel bars 6a, 6b supported in about horizontal position by 4 pieces of stay 7 and the walking floor face 5.

The walker 1, whose accuracy of walking, degree of recovery and effects of rehabilitation, etc. are to be evaluated, walks as straight as possible along the center line 11, toward the image fetching unit 2, on the walking floor face 5.

At that time, the image fetching unit 2 fetches the walking behavior of the walker 1, and electronically transmits that image to the image processing system 3.

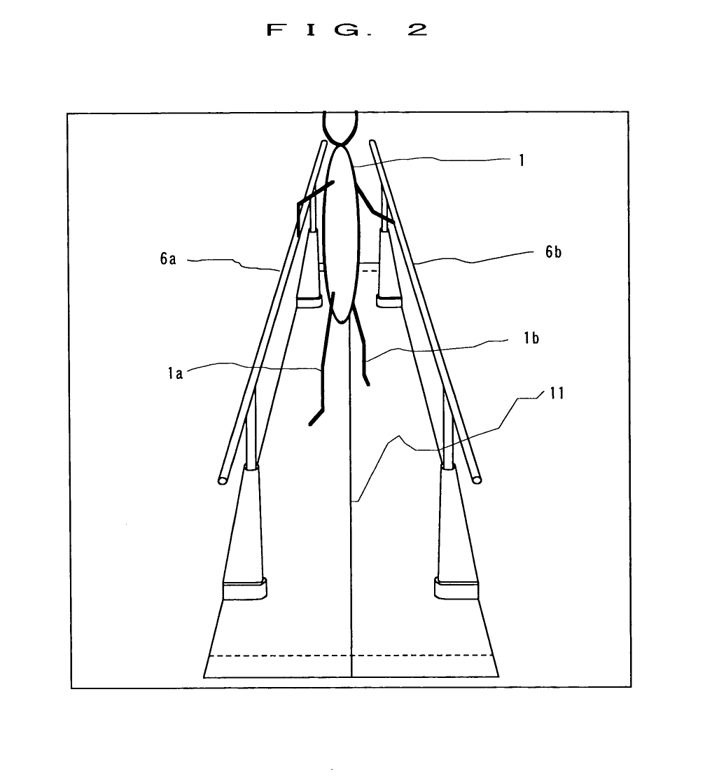

This image signal is given as shown in FIG. 2 for example, and arranged in such a way that at least the right leg 1a and the left leg 1b of the walker 1 exist (appear) in the picture without fail.

Next, on the image processing system 3, the existence of the walker 1 as moving body can be grasped easily by comparison with the previous picture, and the existence of the position of the respective legs can be grasped easily by sequentially scanning from the bottom side of the picture.

Furthermore, the behavior of the left leg 1b and the right leg 1a can be grasped easily by discriminating them from each other, from such conditions that, with an act of walking, the foot positions of the left leg 1b and the right leg 1a alternately move in the downward direction of the picture without fail, the foot position on the opposite side is at stop while one of the legs is moving, the foot position is generally located at the lowest side of the walker in a picture, the right leg 1a is located on the left side of the picture, the left leg 1b is located on the right side of the picture, the left leg is positioned on the right side than the right leg in the picture, the right leg is positioned on the left side than the left leg in the picture, etc.

Still more, during a walk, it never happens that both legs move simultaneously but, when one leg started moving, the leg on the opposite side is sure to be at stop, and one can therefore operate the foot position of the leg at stop.

Yet more, the difference between a moving image (moving leg) and a stationary image (leg at stop) can be easily recognized by constantly making a comparative operation with the previous picture and the subsequent picture.

However, considering that, even if one leg is fixed, the ankle, etc. makes delicate motions, it is possible to ignore such delicate motions and take only the images that move beyond a certain fixed position as moving images.

In addition, by defining, on the image processing system 3, the position from the depth to the front part (from top to bottom) of the picture as coordinate X, and the left-to-right direction as coordinate Y, and checking at which position of the picture the respective foot positions exist, one can easily know the coordinate X and the coordinate Y of the respective foot positions on the floor face 5.

The operation of foot positions described above is not restricted to above, but the foot positions may also be determined by attaching a marker at the foot position and combining the detection of position by this marker and a floor reaction signal (devices) provided on the floor face, etc.

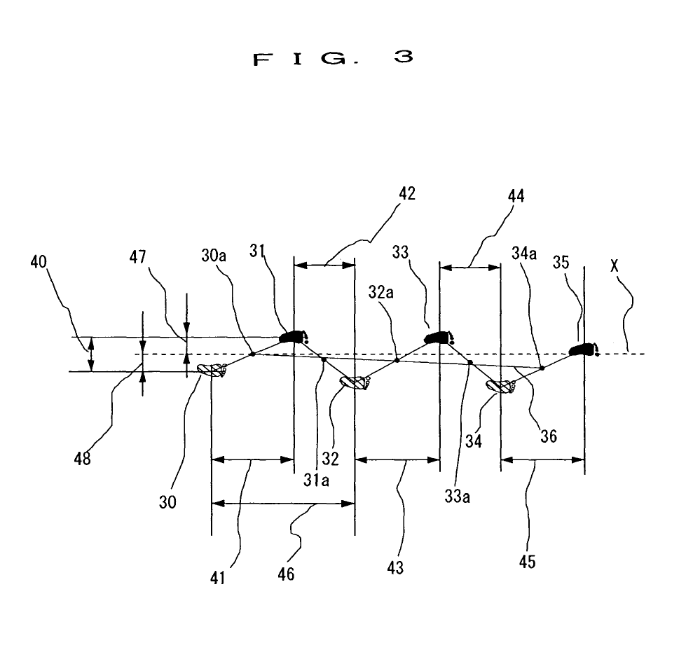

The positions of the respective legs of the walker, which became clear from the above-described analysis, and the footprints operated from their chronological changes may be plotted on an X-Y plane as shown in FIG. 3, for example. Here, understanding will become much easier if different colors are used to show the left and right legs, or the left leg and the right leg are discriminatively indicated in their natural forms as shown in FIG. 3.

Here, the required contents of operation of walking include distinction between the left and right legs, coordinate positions of right leg 30, 32, 34, coordinate positions of left leg 31, 33, 35, step interval 40 between the left and right legs, step 41, 43, 45 of left leg, step 42, 44 of right leg, stride 46, middle point coordinate 30a between 30 and 31, middle point coordinate 31a between 31 and 32, middle point coordinate 32a between 32 and 33, middle point coordinate 33a between 33 and 34, middle point coordinate 34a between 34 and 35, 36 approximating 30a, 31a, 32a, 33a, 34a by either polygonal line or straight line, and non illustrated walking speed, etc.

The stride 46 is obtained by adding the step 42 of the right leg to the step 41 of the left leg.

By removing either the chronological change of steps 47, 48 from the center line X to the respective footprint positions indicated with broken line, or 36, one obtains swing data. Namely, one can easily grasp at a glance if the direction of advance of the walker turns to the left or to the right, if he/she is walking straight, if there is any deviation to left and right even with straight walking, etc.

As described above, the step interval 40, step 41 of left leg, step 42 of right leg, stride 46, center position 30a, deviation 36, walking speed, etc. obtained as a result of operation are classified into momentary values, average values, etc. and stored and recorded in the storage unit of the image processing system 3.

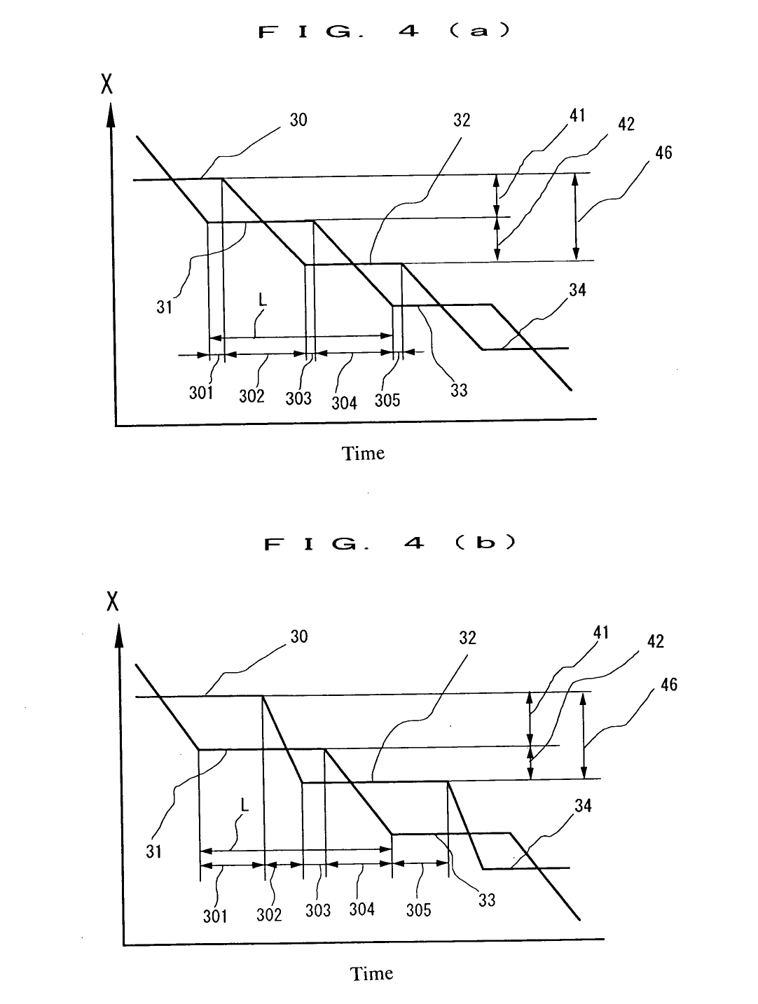

FIG. 4 (a) is an example showing the moving position of legs in a stable and normal walking situation of a sound person, the axis of abscissa indicating the time passed, and the axis of ordinate showing the situation of change in the position X of the respective legs operated by the image processing system 3.

In the walking of a person, the legs can be separated into a landing leg and a moving leg without fail, because the walker puts forward his/her legs alternately.

Even when a leg is landed on the floor face, its ankle is making delicate motions, and if such slight motions are ignored, there exist areas 30, 31, 32, 33, 34, etc. where the legs stay motionless against the time passed as shown in this drawing. And, those areas indicate the foot positions at landing (called base position), and between them follow the data of the position of the moving leg (called free leg position). Namely, 30, 32, 34 indicate the footprints (base position) of the right leg, while 31, 33 indicate the footprints of the left leg.

At that time, the distance 41 between 30 and 31 is the step of the left leg, and the distance 42 between 31 and 32 is the step of the left leg, and the sum 46 of the two is the stride. The walking speed can be calculated easily from the general inclination of all those data.

Moreover, 301 indicates a time range during which both legs remain in contact with the floor face, and is called both-leg supporting period. 302 indicates a time range during which the left leg 31 is in contact with the floor face and the right leg is moving, and is called single left-leg supporting period. 303 is a both-leg supporting period, and 304 indicates a time range during which the right leg 32 is in contact with the floor face and the left leg is moving, and is called single-right leg supporting period. 305 is a both-leg supporting period.

For example, by putting as 301+302+303+304=L, L corresponds to one cycle of the left leg, and 301/L or 303/L are called both-leg supporting periods. In addition, 302/L or 304/L are called single-leg supporting periods.

In a stable and smooth walking of an ordinary sound walker, this both-leg supporting period represents approximately 10% and the single-leg supporting period approximately 40%.

In an actual walking, those data are variable for each step and, for that reason, it is better to calculate the average value for indication. The distances 40, 47, 48 between the left and right legs, etc. can be calculated easily from the Y value at the landing position of both legs.

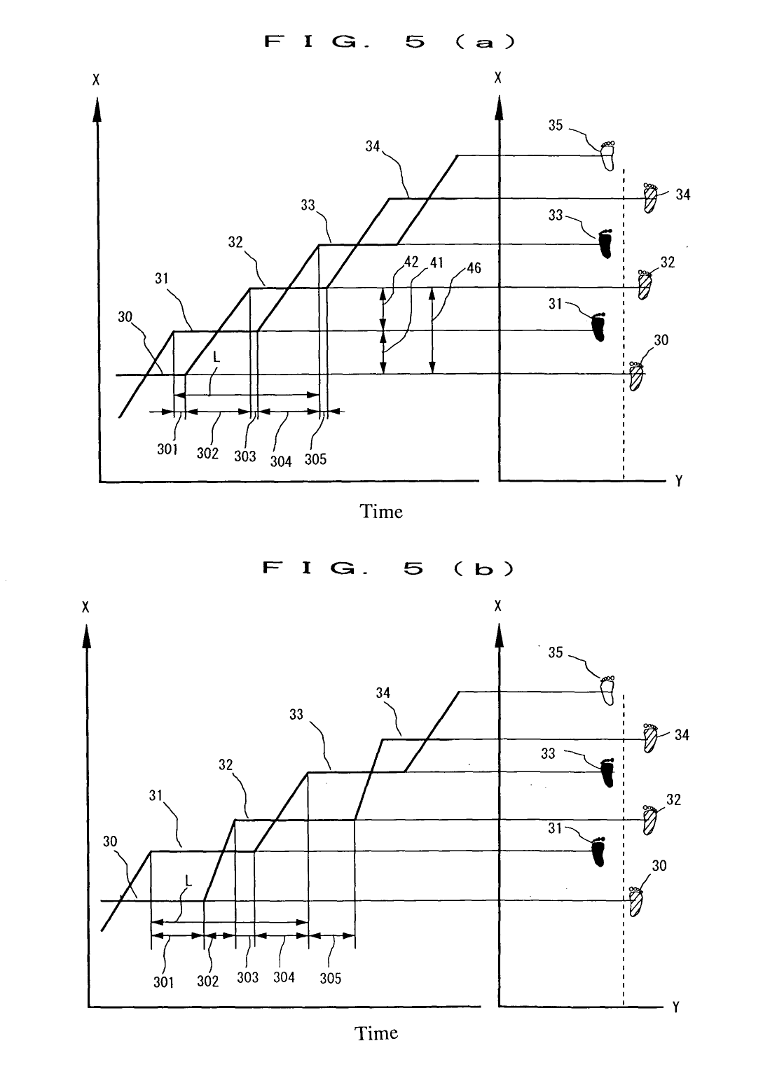

FIG. 5 (a) indicates the contents of FIG. 4 (a) by showing the axis of ordinate upside down, and indicates the foot positions on X-Y coordinates, by putting the axis of abscissa as Y on the right side. Here, one same symbols are applied to the respective symbols which are identical. In addition, in the indication of foot positions, the right leg 30, 32, 34 is hatched, while the left leg 31, 33 is given in black, to clearly show the foot position and the distinction between of the left and right legs. Moreover, the leg position 35 indicates the free leg position.

By indicating as described above, it becomes possible to show both walking motions in time and foot positions in space at a time, in a way very easy to understand for both the walker and the supporting physical therapist.

On the other hand, FIG. 4 (b) is an example showing the situation of walking of a person who is handicapped with the left leg like a patient paralyzed on the left half, etc. and cannot put forward his/her right leg at a large stride (small right step 42) and has a short free leg time (302) of the right leg, the axis of abscissa indicating the time passed, and the axis of ordinate showing the situation of change in the position X of the respective legs operated by the image processing unit.

In this case too, one can see that, while the method of operation and the symbols of results are common to those in FIG. 4 (a), the amount of the step 42 of the right leg is smaller compared with the amount of the step 41 of the left leg.

Furthermore, because the walker cannot stand a long time with the left leg only, the single left-leg supporting period 302 is shorter than the single right-leg supporting period 304. In that case, the length of the both-leg supporting periods 301, 303 does not agree with each other, and the percentage of this disagreement is higher than 10% of sound persons. In the same way, the length of the single-leg supporting periods 302, 304 does not agree with each other, and is extremely shorter compared with 40% of sound persons. Therefore, the walking style is rather clumsy and lacks smoothness, and this seems to imply difficulty of any long time of walking.

FIG. 5 (b) indicates the contents of FIG. 4 (b) by showing the axis of ordinate upside down, and indicates the foot position on X-Y coordinates, by putting the axis of abscissa as Y on the right side. The functions and actions are the same as in FIG. 5 (a).

As described above, defective points or smoothness or stability of walking, etc. become clear from the data on imbalance between the left and right steps and the proportion of single-leg supporting period, and the degree of recovery of walking, etc. also comes to be known by comparison with past data, etc., presenting useful data for the decision of treating policies for the future and for the diagnosis of walking.

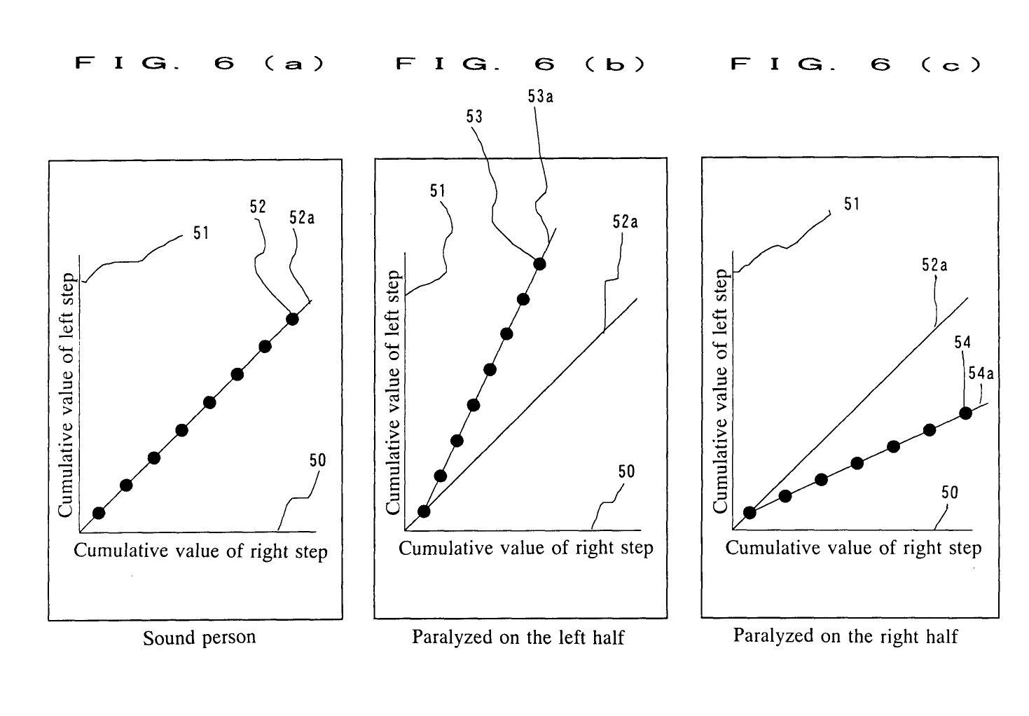

FIG. 6 shows an example in which such data are operated and displayed from a different angle. In the drawing, the axis of abscissa 50 shows a cumulative value of distance of the right step 42, 44 for the right leg only, and the axis of ordinate 51 indicates a cumulative value of distance of the left step 41, 43, 45, etc. for the left leg only, while the black points 52, 53, 54, etc. represent footprint positions plotted on a coordinate graph as coordinate for each of the left and right steps from the first step to the “n”th step.

Still more, in FIG. 6, (a) indicates a general example of walking of a sound person, (b) of a person paralyzed on the left half, and (c) of a person paralyzed on the right half.

In the case of a sound person in FIG. 6, (a), the black points 52 are positioned on a straight line (reference coordinate) 52a inclined by 45 because the left step and the right step are equal to each other.

On the contrary, in the case of paralysis on the left half in FIG. 6 (b), the cumulative value of the right step gradually slows down in its increase because the right step is smaller compared with the left step, and the black points 53 are positioned on the upper side of the straight line 52a at 45 and are linearly approximated as 53a.

Moreover, in the case of paralysis on the right half in FIG. 6 (c), the cumulative value of the left step gradually slows down in its increase because the left step is smaller compared with the right step, and the black points 54 are positioned on the lower side of the straight line 52a at 45 and are linearly approximated as 54a.

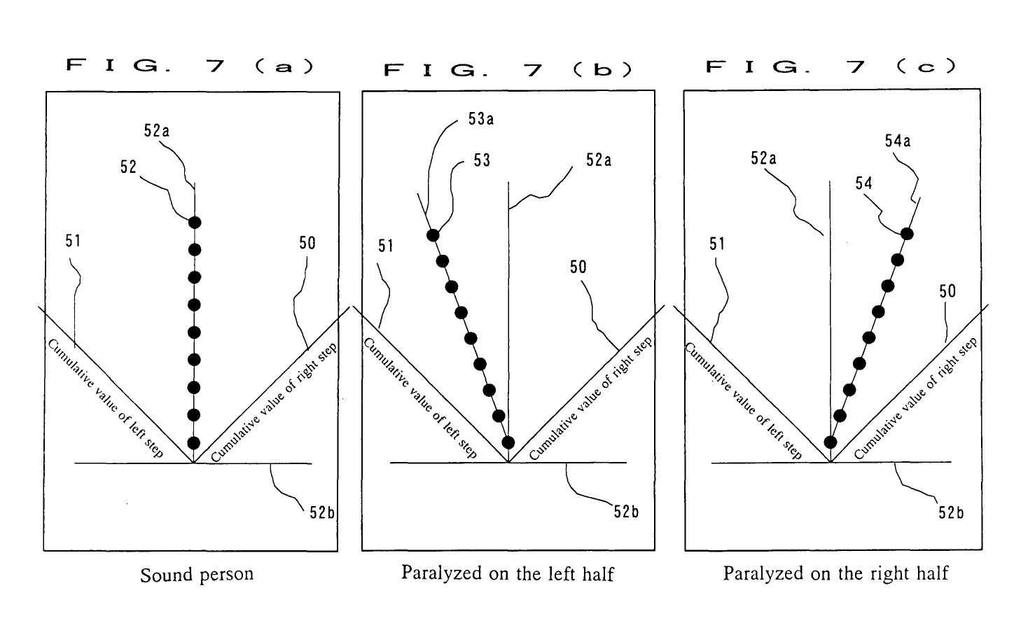

FIG. 7 indicates a case where the respective drawings of FIG. 6 are indicated by turning by 45 in the counterclockwise i.e. left direction.

Therefore, with reference to the axis of ordinate 52a in this FIG. 7, while the black points are positioned on 52a in the case of a sound person (a), the black points are positioned on the left side of 52a in the case of a person paralyzed on the left half (b), and on the right side of 52a in the case of a person paralyzed on the right half (c), and this makes it possible to know at a glance which leg is unusual and what is the degree of abnormality compared with a sound person, and to also grasp the achievements of exercise or degree of recovery, etc. through comparison with past data.

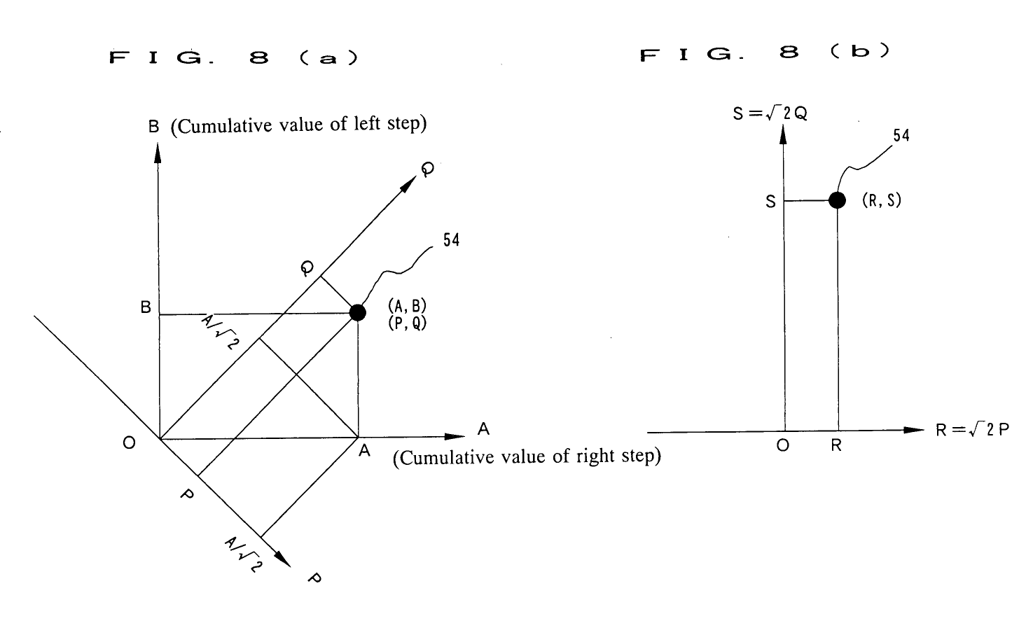

FIG. 8 is an explanatory drawing of a case where FIG. 7 is converted into actual physical quantities, defining the axis of abscissa in (a) as A (cumulative value of right step 50), the axis of ordinate as B (cumulative value of left step 51), diagonal axes at 45 as axis P, axis Q respectively, and the coordinate values of walking data string 54 as (A, B) on the A, B orthogonal coordinates or as (P, Q) on the P, Q orthogonal coordinates.

Consequently, from FIG. 8 (a), one obtains geometrically

P=A/\sqrt{2}−B/\sqrt{2} (1)

Q=A/\sqrt{2}−B/\sqrt{2} (2)

Furthermore, FIG. 8 (b) indicates the axis of abscissa as R=\sqrt{2P} and the axis of ordinate as S=\sqrt{2Q}, or, in other words, the results of turning by 45° the entire drawings in FIG. 8 (a) in the counterclockwise direction by defining the axis of abscissa as P and the axis of ordinate as Q and further expanding them to \sqrt{2} times the initial size.

Consequently, from said formulas (1), (2), the coordinates (R, S) of walking data 54 are given as:

R=A−B (3)

S=A+B (4)

Namely, the axis of abscissa R represents the cumulative value of the differences between the right step and the left step, the axis of ordinate S shows the cumulative value of the right step+the left step i.e. the respective strides, and therefore the position of advance of the walker.

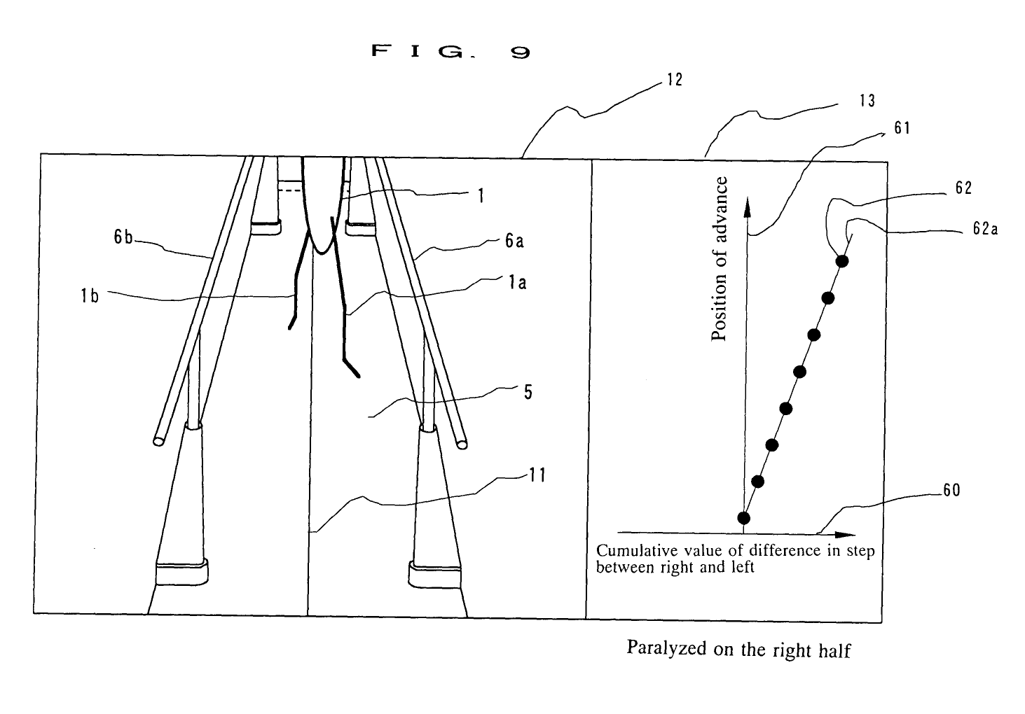

FIG. 9 indicates an example of the contents displayed on the display unit 4 during a walk. In this drawing, 12 indicates the image fetched by the image fetching unit, and 13 indicates the results of operation of FIG. 8 (b) respectively.

Those indications can be displayed and recorded while operating about in real time during a walking exercise, with the use of a personal computer, etc.

The image 12 here is displayed in such a way that the respective pixel information of the fetched image is once fetched into the storage unit of the image processing system 3, to be shown in a way inverted in the left-right direction i.e. as a mirror image, on the display.

As the scope of display, the entire part of the fetched image in FIG. 2 may be displayed by inversion in the left-right direction, or by expanding centering around the walking face as shown in FIG. 9.

Moreover, in FIG. 9, while the results of operation 13 are given for a case of paralysis on the right half, the contents of operation in FIG. 7 are multiplied by \sqrt{2} for both the axis of abscissa and the axis of ordinate, as explained above, and the axis of abscissa 60 (right-left) indicates the cumulative value of differences in step, while the axis of ordinate 61 represents the position of advance of the walker, and this makes it possible to verify and judge instantly which leg is handicapped and cannot make a normal step, by simply checking visually the trend of the walking footprint data 62, namely on which side of the position of advance 61 the linear approximation 62a is inclined.

Furthermore, by checking deviation from the linear approximation 62a at the same time, one can also check stability easily.

This display unit is provided in about the front direction of the walker, as shown in FIG. 1, making it possible for the walker 1 to take walking exercise while watching the displayed results in FIG. 9. By walking while visually checking with his/her own eyes his/her own walking posture, namely inclination of legs and knees, inclination to left and right or balance of waist in some cases, the walker can take walking exercise in the state looking into the front direction, without looking downward.

Here, the displayed image 12 in FIG. 9 is given in a state inverted in left-right direction, the right leg 1a is displayed on the right side of the picture, and the left leg 1b is shown on the left side of the picture.

Therefore, the walker can understand his/her own walking posture and visually check it with his/her own eyes, without any deep thinking in the head, and can thus take walking exercise while correcting any imbalanced part or inclination.

The display unit 4, which can be easily moved if it is a display unit like personal computer, etc., shall most preferably be installed at the height of the walker's eyes, to make it possible for the walker to visually control or correct his/her own walking posture in a state maintaining a correct walking posture.

Moreover, for walkers with a weak sight, a large projector, etc. may be used as display unit, to show the image by extending it to a desired size with a computer.

By the way, for inverted indication in left-right direction, there is a system in which a mirror is installed vertically in the front face of the walker and the walker takes walking exercise while watching his/her own walking posture reflected in the mirror, but no expanded view can be displayed in the case of a mirror.

Furthermore, though expansion is possible with the use of a concave mirror, it is impossible to change the magnification and it may also cause distortion of the floor face. In addition, in the case of a mirror, the mirror is installed in vertical position normally to avoid inclination of the floor face, but this has a defect of not allowing the walker to take his/her posture while looking correctly into the front direction, because the feet which the walker wishes to visually check with special interest come to the bottom side.

Since these pictures 12, 13 can be operated and displayed in real time in line with the walking practice, the walker can take walking exercise while visually checking these pictures at the same time.

However, the picture 13 on the right is not restricted to above, but may also display the walking footprint as indicated in FIG. 3, or the contents as shown in FIG. 4 to FIG. 6, or display a plural number of those contents at a time.

Still more, it may also display the amount of deviation to left and right or meandering against the center line 11.

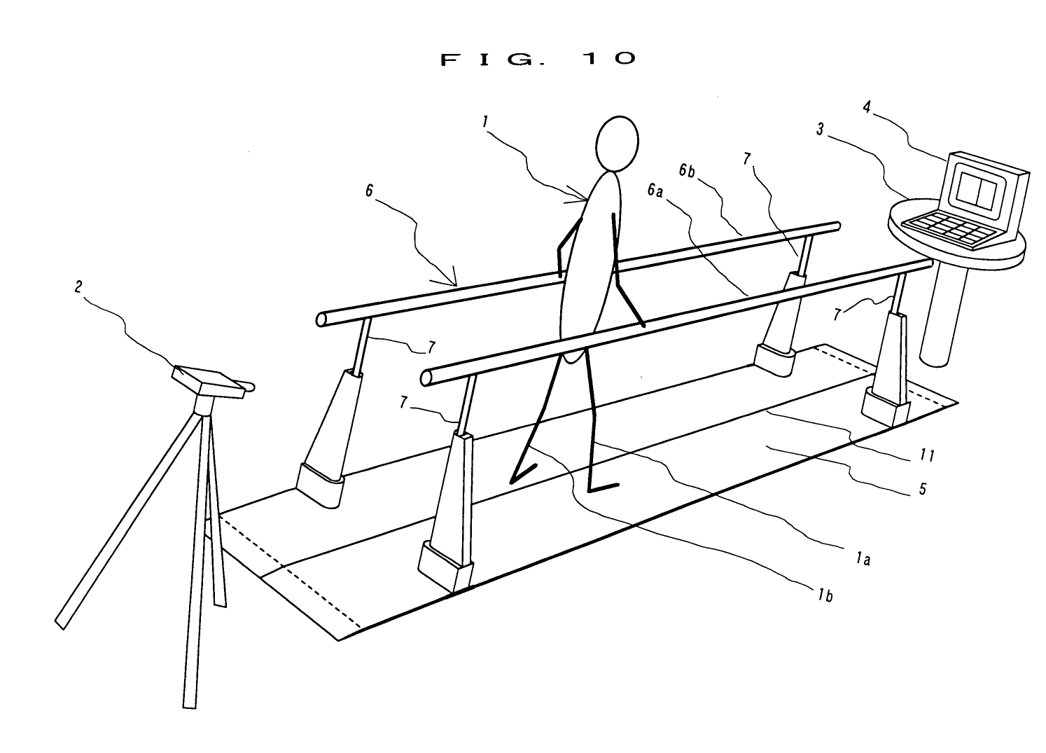

On the other hand, FIG. 10 indicates other embodiment of the present invention, which is different from the embodiment indicated in FIG. 1 in that the image fetching unit 2 is installed at a position in the back face direction and not in the front direction of the walker 1.

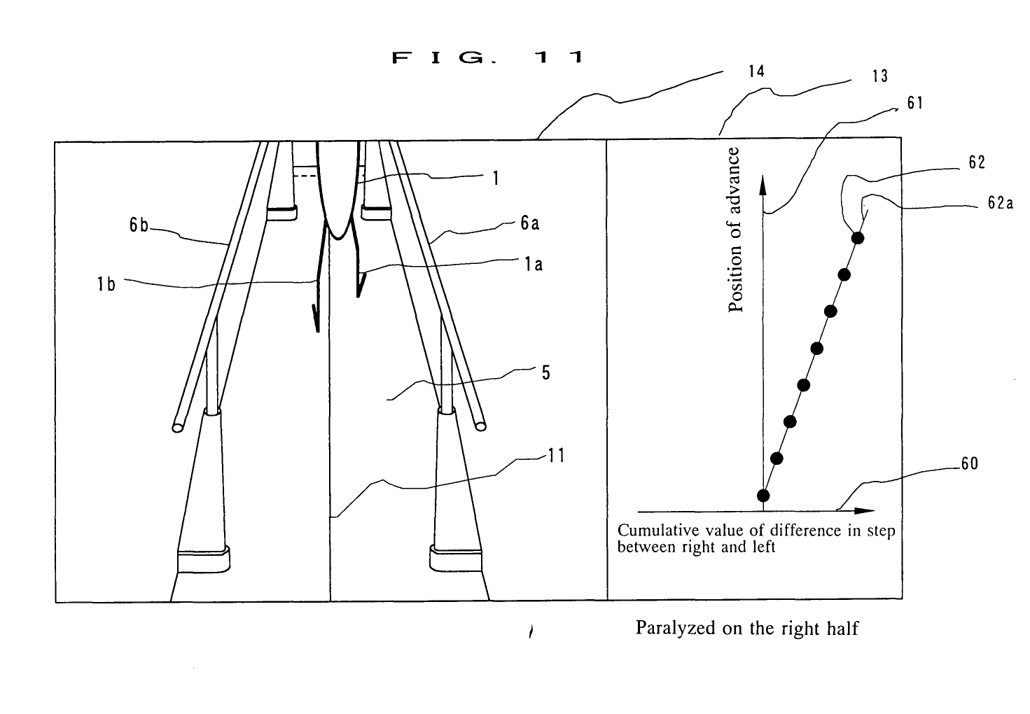

An example of the results of display on the display unit 4 in this case is indicated in FIG. 11.

From this image fetching unit 2, it becomes possible to catch the back view of the walker 1, and show it on the display unit 4 in such a way that the right leg 1a is positioned on the right side and the left leg 1b on the left side as shown on the picture 14 on the left in FIG. 11.

The results of operation 13 on the right side indicated in FIG. 11 are the same as those in the case of FIG. 9, and have the same actions and effects.

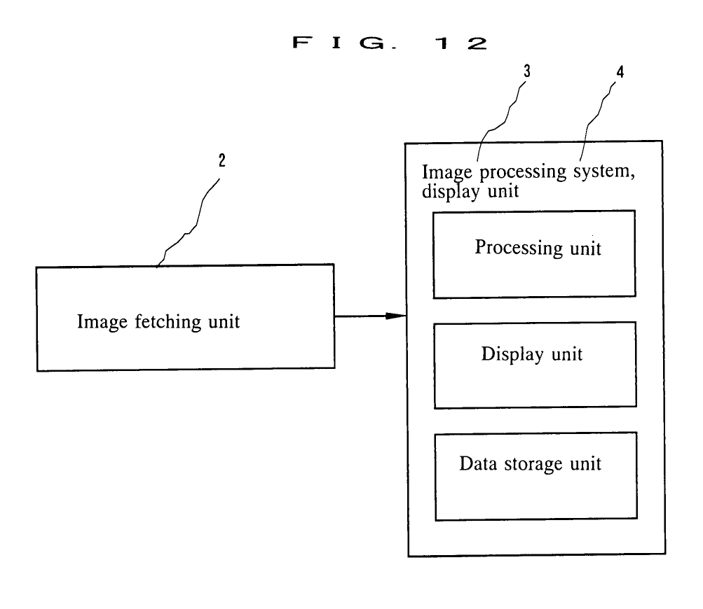

FIG. 12 indicates the flow of signals from the footprint analyzer in said respective embodiments, in which the image signals fetched through the image fetching unit 2 are sent through a non-illustrated image input board to the image processing system 3, to be processed.

The image processing system 3 also has a function of statistical processing such as averaging, etc., and a personal computer, etc. may be used, for example, as such image processing system 3.

The results of operation of the image processing system 3 are indicated on the display unit 4, and those results of operation may also be memorized or read out by means of a storage unit.

Moreover, this storage unit also enables to record, by using personal computer, etc., the exercise data obtained in the image processing system 3 in portable recording media such as floppy disc, etc.

Furthermore, the display unit 4 also enables to indicate chronological fluctuation values of the results of operation, or make comparative display with past exercise data read out from recording media such as floppy disc, etc.

Still more, it further enables to make comparison with the results of past exercise, playback, etc. and the results of such comparison or playback are sent to the display unit 4, for indication.

As described so far, according to the footprint analyzer of this embodiment, said image processing unit discriminates the left and right legs from said fetched image and calculates the floor face contact position of the respective legs and chronological changes of each leg at the floor face contact position, while said display unit displays the floor face contact position of the respective legs and chronological changes of each leg and, for those reasons, the walker can walk, while recognizing the behavior of his/her left and right legs and visually checking, with his/her own eyes, his/her own walking posture, namely inclination of legs and knees, inclination to left and right or balance of waist, etc. in some cases, and can also take walking exercise in the state looking into the front direction, without looking downward, by installing the display unit at a proper position.

Here, the displayed image is given in a state inverted in left-right direction in the case of photographing from the front face but in a state not inverted in left-right direction in the case of photographing from the rear face and, for that reason, the right leg is displayed on the right side of the picture, and the left leg is shown on the left side of the picture. Therefore, the walker can understand his/her own walking posture and visually check it with his/her own eyes, without any deep thinking in the head, and can thus take walking exercise while correcting any imbalanced part or inclination.

The display unit 4, which can be easily moved if it is a display unit like personal computer, etc., may be installed at the height of the walker's eyes, thus making it possible for the walker to visually control or correct his/her own walking posture in a state maintaining a correct walking posture. Moreover, for walkers with a weak sight, a large projector, etc. may be used as display unit, to show the image by extending it to a desired size with a computer.

Yet more, it is possible to perform operation or statistical processing, display and storage of the walker's walking speed, distance between the steps, distance of step between the left and right legs, condition of staggering to left and right, etc. In addition, it becomes possible to quantitatively grasp the situation of progress of the exercise and the degree of recovery, etc., through comparison with past data, etc., and this enables doctors and physical therapists to effectively promote walking exercise of the patients, by effectively utilizing the results together with said data, and realize a short-period walking exercise.

As described above, the footprint analyzer of the respective embodiments can perform operation or statistical processing, display and storage of walking speed, distance between the steps, distance of step between the left and right legs, condition of staggering to left and right, etc. in real time and, by effectively utilizing such data for treatment and diagnosis in the future, it becomes possible for doctors and physical therapists to effectively promote walking exercise of the patients. As a result, it becomes possible to implement training in a short time.

The footprint analyzer according to the present invention has so far been explained based on its embodiments, but this invention is not restricted to said embodiments but may take different constructions as required, to an extent not deviating from its main purpose, by enabling to display the left and right coordinate axes in inverted way, as required, in FIG. 6 to FIG. 8, for example.

1. A footprint analyzer comprising an image fetching unit for retrieving walking images, an image processing unit for treating the images from the image fetching unit, a display unit for displaying the results of the operation of the image processing unit, and a storage unit for saving the operation results, characterized in that said image processing unit discriminates the left and right legs from said fetched images and calculates the floor face contact position of the respective legs and chronological changes of each leg at the floor face contact position, while said display unit displays the floor face contact position of the respective legs and the chronological changes of each leg.

2. A footprint analyzer as defined in claim 1, wherein said image processing unit performs operation or statistical processing of the walker's walking speed, the distance between the left and right legs, the distance between the front and rear legs, the distance between the front and rear positions of each leg, any staggering to the left or right, the support rate of both legs and/or the support rate of a single leg, and said display unit displays part or all of the operation results.

3. A footprint analyzer as defined in claim 1, wherein said storage unit memorizes the walker's symbol, operation results or the results of statistically processing the walking speed, the distance between the left and right legs, the distance between the front and rear legs, the distance between the front and rear positions of each leg, any staggering to the left or right, the support rate of both legs and/or the support rate of a single leg of the walker at each time of measurement, and said image processing unit performs a comparative operation of data for each measurement.

4. A footprint analyzer as defined in claim 1, wherein said display unit is provided in front of the walker.

5. A footprint analyzer as defined in claim 1, wherein the image processing unit accumulates the right step on one of the coordinate axes of two-dimensional coordinates, accumulates the left step on the other axis of coordinate, determines the coordinates of the respective steps, and indicates the coordinates on a coordinate graph.

6. A footprint analyzer as defined in claim 1, wherein the image processing unit displays images by turning them at required angle, so that the standard coordinates of a sound person may be given on the axis of ordinate.

7. A footprint analyzer as defined in claim 1, wherein the image processing unit displays the center locus for the respective position of each leg.
